(19) United States
(12) Patent Application Publication
(10) Pub. No.: US 2017/0326154 A1
(43) Pub. Date: Nov. 16, 2017

(54) Pre-Operative Composition for Sedation or Analgesia

(71) Applicant: Stephen Parker, Kalamazoo, MI (US)

(72) Inventor: Stephen Parker, Kalamazoo, MI (US)

(21) Appl. No.: 15/592,189

(22) Filed: May 10, 2017

Related U.S. Application Data
(60) Provisional application No. 62/334,315, filed on May 10, 2016.

(51) Int. Cl. A61K 31/5517; A61K 31/4174; A61K 45/06; A61K 47/40

(52) U.S. Cl. CPC A61K 31/5517; A61K 47/40; A61K 31/4174; A61K 45/06

(57) ABSTRACT

A pre-operative composition for sedation or analgesia is used to sedate a patient before a medical procedure. The pre-operative composition is formulated to prevent negative effects felt by the patient after the medical procedure. The pre-operative composition includes dexmedetomidine, midazolam, and some kind of pharmaceutical carrier. The pre-operative composition can be administered in a variety of methods to the patient. The preferred method of administering the pre-operative composition is either orally or by a syringe.

The current application claims a priority to the U.S. Provisional Patent application Ser. No. 62/334,315 filed on May 10, 2016.

### FIELD OF THE INVENTION

The present invention generally relates to a pharmaceutical composition that has been premixed with multiple active ingredients. More specifically, the present invention is a pre-mixed pharmaceutical composition containing midazolam and dexmedetomidine in prefilled delivery system.

### BACKGROUND OF THE INVENTION

Racemic 4-[1-(2,3-dimethlyphenyl)ethyl]-1H-imidazole, which is known under the name medetomidine, is a selective alpha-2-adrenoceptor agonist. Medetomidine has been used as an antihypertensive agent and as a sedative/analgesic agent and anesthetic adjuvant. It has further been observed that this compound possesses anxiolytic effects and can therefore be used in the treatment of general anxiety, panic disorder, and various types of withdrawal symptoms. Additional effects related to anesthetic care include antisialogogue effects, pediatric emergence delirium preventative/treatment effects, neuro-protective effects, absence of post-operative nausea and vomiting (PONV), narcotic-sparing effects, increased hemodynamic stability of the patient/subject during the peri-procedural/peri-operative experience, decreased gastric secretion effects, absence of respiratory depressive effects, decreased cerebral oxygen consumption effects, absence of delays in post-operative discharge or increased length of stay (LOS), and effects relating to the reduction in activity of the locus coeruleus (LC) in the central nervous system (stimulation of which by agents utilized to induce and/or maintain general anesthesia is thought to cause emergence delirium/pediatric emergence delirium).

The d-enantiomer of the medetomidine, the generic name of which is dexmedetomidine, is an alpha-2-adrenoceptor agonist for general sedation/analgesia and the treatment of hypertension or anxiety. Dexmedetomidine may be used in perioperative settings and by the epidural route. For example, when used in perioperative care, dexmedetomidine can reduce the amount of anesthetic(s) necessary to anesthetize a patient/subject. Additionally, the use of dexmedetomidine in treating glaucoma and the use of dexmedetomidine for preventing neurodegeneration caused by ethanol consumption are known. Furthermore, dexmedetomidine, or a pharmaceutically acceptable salt thereof, is utilized for sedating a patient/subject in an intensive care unit.

Dexmedetomidine can be administered to a patient/subject by numerous methods. For example, administration of dexmedetomidine via parenteral, intravenous, and oral routes; including intramuscular and intravenous administration, administering dexmedetomidine through the skin, etc. Additionally, dexmedetomidine can be administered transmucosally.

Dexmedetomidine has historically been provided as a concentrate that must be diluted prior to patient administration. The requirements of a dilution step in the preparation of the dexmedetomidine formulation is associated with additional costs and inconvenience, as well as the risk of possible contamination or overdose due to human error. Newer formulations are provided in premixed vials/bottles at volumes in excess of many patient/subject requirements during one-time administration or infusion. Thus, a dexmedetomidine formulation that avoids expense, inconvenience, delay, risk of contamination or overdose, and reduces drug waste would provide significant advantages over currently available formulations. Furthermore, current formulations are unpalatable orally and onset of effects requires extensive time.

8-chloro-6-(2′-fluorophenyl)-1-methyl-4H-imidazo[1,5-a][1,4]-benzodiazepine is a short-acting, potent sedative/hypnotic agent with anxiolytic properties. It possesses an excellent safety profile with minimal cardiovascular and respiratory side effects. Its unique pharmaceutical profile makes it useful in pre-operative sedation.

Conscious sedation techniques utilizing midazolam, or in combination with other agents, have become increasingly popular for a wide array of surgical procedures, especially those of short duration. This allows for the avoidance of general anesthesia techniques, which is advantageous for health and financial reasons. Pre-operative management of young/children subjects/patients is often difficult related to separation anxiety from parents/guardians/care-givers and the strange, foreign healthcare/hospital/surgical environment(s). The utilization of sedative agents decreases anxiety and minimizes psychological trauma.

Midazolam anesthetic benefits include a relatively short duration of action, a relatively quick time to peak effect when administered orally, may be administered orally via palatable solutions/suspensions, provides anxiolysis and retrograde amnestic effects, possible prevention of pediatric emergence delirium, statistically significant improved post-surgical night 1 sleep with avoidance of sleep disturbances (including, but not limited to, bed-wetting, night terrors, and increased anxiety), and a proven degree of safety, with little respiratory and cardiovascular effects and the availability of a reversal agent.

Midazolam is approved for intravenous and/or intramuscular injection in adults, and oral administration via tablets. Oral administration is preferred to other routes as it removes possible pain and distress. Conversely, the oral route is disadvantageous due to pre-systemic metabolism and delayed onset of action compared to parenteral routes. Complications may also exist related to increased consumed volumes of material present/remaining in the stomach or gut.

Oral pre-medication is popular in young/child subjects/patients for sedation. Unfortunately, the relatively slow onset of action requires careful timing of administration. Furthermore, due to delayed gastric emptying and uptake during general anesthesia, volumes may persist in the stomach/gut post-surgically; causing increased sedative effects and delaying emergence/awakening and discharge, and also increasing length of stay. Additionally, midazolam onset orally is relatively slow versus other methods and children may spit or regurgitate the administered medication, while others may refuse to accept the medication or may be unable to receive it due to vomiting. In larger administered oral volumes of midazolam, the risk of aspiration becomes unacceptable due to the volume administered and sometimes from the patient/subject's belief that by taking oral medications it is now acceptable to drink and/or eat other liquids and/or foods. Oral formulations currently available are relatively palatable, due to the addition of sweeteners, natural and/or synthetic, and flavorings, natural and/or synthetic. The nasal route has been explored as a convenient, reliable, and acceptable alternative resulting in rapid entry into central nervous system tissues resulting in rapid action.

Transmucosal routes of delivery via the oral and/or nasal cavities offer a means of direct drug delivery to circulation versus the disadvantages of orogastric administration. Midazolam is a basic drug, resulting from the nitrogen atom in the imidazole ring fused to the benzodiazepine skeleton. The pKa value ranges between 5.5 and 6.2 with a highly pH-dependent water solubility. Midazolam has increased solubility at low pH results from ionization. At or below a pH of 4, part of the drug will present with an open benzene ring bearing a highly ionizable primary amino group, imparting water solubility. At or above a pH of 5, the majority of the drug presents with a ring closed form, resulting in increased lipid solubility.

Midazolam solutions have been utilized in numerous clinical investigations via intranasal, sublingual, and rectal routes. Oral administration often results in adequate sedation within 10-20 minutes whereas intranasal administration results in adequate sedation within 10-15 minutes but fails due to intense burning, irritation, and discomfort. The failures of intranasal administration are attributed to the low pH of the solution, approximately 3.2 (versus 5.5-6.6 nasal pH), required to maintain midazolam in the solution. This has resulted in failure to gain approval for the intranasal route. Sublingual administration results in adequate sedation similar to intranasal administration but may suffer from poor taste and the requirement of relatively large volumes resulting from drug spreading and swallowing. Significant amounts of the drug may be lost to ejection resulting from poor taste encountered by younger patients/subjects.

An intranasal administered form of midazolam hydrochloride may be prepared by maintaining a pH of 3.62. It suffers the same disadvantages previously described and contains midazolam in a predominantly open ring form. Additionally, available forms of midazolam include buccal and sublingual tablets/lozenges, adhesive patches, gels, solutions, or sprays (powder, liquid, or aerosol) for the oral cavity, and solutions or sprays (powder, liquid, or aerosol) for the nasal cavity. Resulting from poor transport/uptake of drugs across membranes when administered via mucosal routes, few drugs are available per these routes.

Orally administered midazolam has historically been provided as a solution/suspension in a multi-dose container/vial that must be withdrawn from the container/vial prior to patient administration and then re-stored for later use. The requirements of this step are associated with additional costs and inconvenience, as well as the risk of possible contamination or overdose due to human error. Thus, a midazolam formulation that avoids expense, inconvenience, delay, risk of contamination or overdose, and reduces drug waste would provide significant advantages over currently available formulations. Furthermore, current formulations are only moderately palatable orally and onset of effects requires moderate to extensive time.

Currently, inadequate agents exist to prevent pediatric emergence delirium (PED) in a prophylactic manner. Modern pre-operative agents provide sedation or analgesia, with varying degrees of effectiveness, but are highly inadequate to prevent PED occurrence in the post-anesthesia care unit (PACU), also known as the recovery room/area. Furthermore, these agents significantly delay awakening from anesthesia, delay discharge, and increase length of stay (LOS). These current agents may be administered orally (PO), the preferred route, or by the intravenous (IV), intramuscular (IM), inhalational (INH), rectal (PR), oral-gastric tube (OG), oral-jejunal tube (OJ), or intranasal (IN) routes, with varying degrees of absorption/elimination, comfort/discomfort, tolerance, effectiveness, and acceptability.

PED occurs immediately after post-procedural emergence from inhalational, and possibly intravenous, anesthetic agents/general anesthesia. It is characterized by restlessness, incoherence, and inconsolability. It may be accompanied by thrashing, crying, screaming, and disorientation, that can lead to 1) drain, IV catheter, and other medical device(s) dislodgement, 2) increased pain, and 3) surgical site hemorrhage. Generally reported rates of occurrence are 2-55%, with some as high as 10-80%. These behaviors may result in prolonged post-anesthetic recovery, increased nursing/medical care, restraint, delayed familial reunion, inpatient admission, and increased costs [incurred by the patient(s) and care center(s)].

PED typically resolves within 30 minutes after emerging from general anesthesia. However, cases lasting over 48 hours have been reported, and are associated with long term sequelae such as new-onset maladaptive behaviors, including eating issues, sleep disturbances/night terrors, separation anxiety, and apathy. Patients reporting to the PACU with an episode of PED are 7 times more likely to develop these maladaptive behaviors and more marked episodes increase the risk. Injuries to healthcare personnel have also been reported. These include, but are not limited to, cuts, scratches, bruises, and abrasions.

Emergence delirium is a phenomenon experienced, but not commonly, by adult patients as well. Furthermore, current rates of patients/subjects reporting to clinical sites with past medical/psychological histories, that include, but are not limited to, post-traumatic stress disorder (PTSD), are increasing. Such patients/subjects often suffer/encounter the same post-anesthetic sequelae as those experiencing PED. Each of these conditions may be prevented prophylactically by the administration of midazolam or dexmedetomidine pre-procedurally. However, each agent retains disadvantages when given independently. Without adequate IV access (as is often commonplace in anxious, needle-shy, and/or mentally or physically delayed and/or challenged adult patients or pediatric/younger patients, of any race or sex) a rapidly administered route with rapid drug absorption/uptake, no pain, palatability, and decreased volume administered is necessary. Furthermore, midazolam suffers from debatable effects in prevention of PED and/or psychological pathologies, delayed discharge, delayed recovery, delayed extubation, and delayed anesthetic emergence, all of which may result in increased costs, increased pain, injury, etc. at currently recommended/administered doses. Dexmedetomidine suffers from poor palatability and increased times to adequate sedation attainment/establishment when administered orally.

Therefore, the objective of the present invention is to combine these two agents as a premixed pharmaceutical composition containing midazolam and dexmedetomidine. The present invention is typically administered in a prefilled, single-use delivery system while adjusting/decreasing doses for synergy of the agents and concentrating the composition to decrease volumes administered orally. Thus, the resulting compound retains the positive benefits of midazolam oral suspension (palatability, short duration of action, oral administration, comfort, pain-free, anxiolysis/amnesia, relatively fast onset, prevention of separation anxiety, possible PED prevention, safety, statistically significant improved post-procedural/post-surgical night 1 sleep with avoidance of sleep disturbances, etc.) and dexmedetomidine solution (antisialogogue effects, known PED prevention, oral administration, analgesia, sedation, neuro-protective effects, no PONV, no delays in discharge/extubation/emergence, decreased LOS, reduced LC activity in the central nervous system, narcotic-sparing effects, decreased gastric secretions, no respiratory depression, increased hemodynamic stability, decreased cerebral oxygen consumption, etc.). These benefits/advantages make a premix pharmaceutical composition containing midazolam and dexmedetomidine into a unique and superior agent that is used in, but not limited to, pediatric surgery/anesthesia. The present invent is the best choice for a patient to get the best outcomes/results, safety, prevention of harm/injury, and surgical outcomes/experiences possible. Additionally, the combined compound results in decreased overall risks of PED and psychological pathology manifestation, decreased separation anxiety and total patient anxiety, increased vital sign stability, decreased risks, increased patient mask acceptance and cooperation during inhalational general anesthetic induction, decreased medication volume administration (decreasing the risk of aspiration), a single agent for administration (resulting in no need for further trauma resulting from repeated medication administrations/insults), decreased narcotic requirements intra- and post-operatively, prophylactic/preventative analgesia, decreased parental/guardian/family anxiety (resulting in further decreased patient/subject anxiety), and dual agent synergy (resulting in decreased dose and volume administration requirements and faster onset/shorter time to achievement of adequate patient sedation/comfort).

### BRIEF DESCRIPTION OF THE DRAWINGS

FIG. 1 is a block diagram illustrating both mandatory and optional constituents of the present invention.

### DETAILED DESCRIPTION OF THE INVENTION

All illustrations of the drawings are for the purpose of describing selected versions of the present invention and are not intended to limit the scope of the present invention.

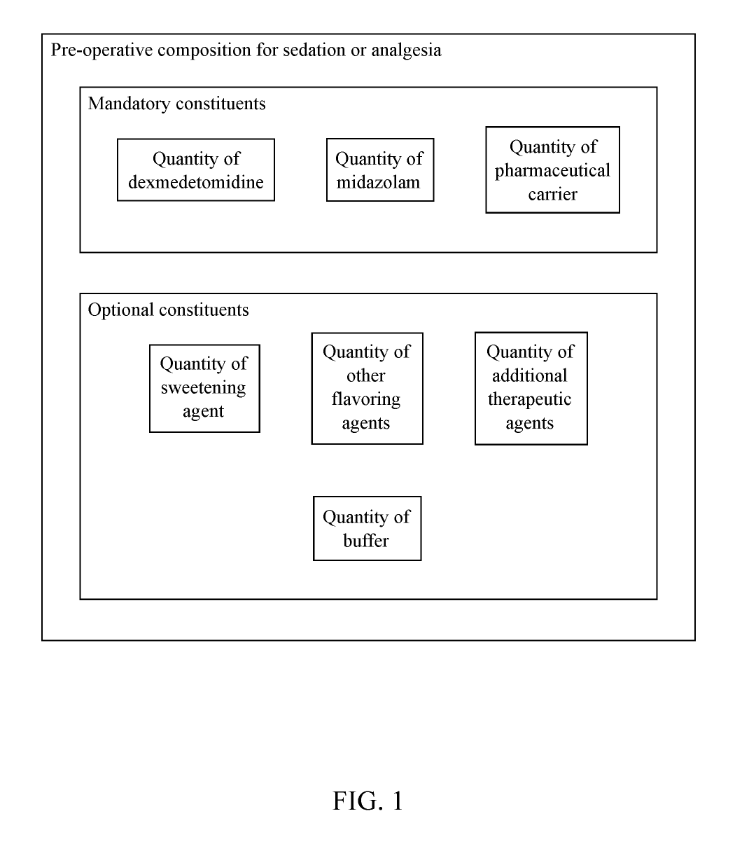

The present invention is a pre-operative composition for sedation or analgesia, which is used to prevent certain post-operative negative effects felt by a patient. As can be seen in FIG. 1, the present invention comprises a quantity of dexmedetomidine, a quantity of midazolam, and a quantity of pharmaceutical carrier. Dexmedetomidine is a medication that is capable of reducing anxiety, relieving pain, and acting as a sedative. Midazolam is a medication that is capable of inducing anesthesia, acting as a sedative, and reducing severe agitation. Together, the quantity of dexmedetomidine and the quantity of midazolam are mixed into the quantity of pharmaceutical carrier in order to create a readily-dispensable form of the present invention. The pharmaceutical carrier is used to efficiently and effectively deliver the dexmedetomidine and the midazolam into a patient. Also in some embodiments of the present invention, the quantity of dexmedetomidine and the quantity of midazolam are formulated as pharmaceutical salts in order to prevent the present invention from becoming too basic. These pharmaceutical salts would preferably be further composed of hydrochloride.

The concentrations of dexmedetomidine and midazolam within the pre-operative composition can adversely affect the patient if those concentrations are either too high or too low. Thus, the quantity of dexmedetomidine ranges from a concentration of 2 micrograms per milliliter (mcg/mL) within the pre-operative composition to a concentration of 50 mcg/mL within the pre-operative composition. In addition, the quantity of midazolam ranges from a concentration of 100 mcg/mL within the pre-operative composition to a concentration of 5000 mcg/mL within the pre-operative composition. The preferred embodiment of the present invention retains the quantity of dexmedetomidine at an approximate concentration of 16 mcg/mL within the pre-operative composition and retains the quantity of midazolam at an approximate concentration of 400 mcg/mL within the pre-operative composition. The preferred embodiment of the present invention is administered PO 10-20 minutes pre-operatively at a dose of 0.25 milliliter per kilogram (mL/kg) of patient weight (a range of 0.15 mL/kg to 0.4 mL/kg) from a pre-filled syringe delivery system including a set of “kg” markings on the syringe in addition to a set of “mL” markings.

The pharmaceutical carrier can be configured in a variety of designs because the active ingredients of the present invention are dexmedetomidine and midazolam. Thus, the pharmaceutical carrier can be, but is not limited to, an aqueous liquid, a gel, or a suspension solution. More specifically, the quantity of pharmaceutical carrier can be made of, but is not limited to, water, ethanol, glycerin, polyethylene, glycol, propylene glycol, or a combination thereof.

The taste of dexmedetomidine and midazolam can be bitter, if the present invention is orally administered to the patient. Thus, the present invention may further comprise a quantity of sweetening agent that is mixed into the quantity of pharmaceutical carrier. The quantity of sweetening agent is preferably at an approximate concentration of 50,000 mcg/mL, which needs to be at a larger concentration in order to effectively counteract the extremely bitter taste of dexmedetomidine and midazolam. In preferred embodiment of the present invention, the quantity of sweetening agent is cyclodextrin. In addition, the present invention may further comprise a quantity of other flavoring agents that is similarly mixed into the quantity of pharmaceutical carrier. The other flavoring agents not only counteract the bitter taste of dexmedetomidine and midazolam but are also able to cater the overall taste of the present invention to each individual patient. The quantity of other flavoring agents can be, but is not limited to, at least one carbohydrate, at least one sweeting agent, at least one flavor enhancer, at least one preservative, at least one antifungal agent, or a combination thereof. Moreover, the quantity of other flavoring agents is used to cater the overall taste of the present invention when the patient is a child so that the present invention can be more easily administered to the child. Consequently, the quantity of other flavoring agents can adjust the overall taste to be a cherry flavor, a grape flavor, an orange flavor, a bubble-gum flavor, a strawberry flavor, or any other flavor that appeals to the child.

A pH level of the pre-operative composition is preferably configured to be greater than or equal to 4. In order to maintain a consistent pH level, the present invention may further comprise a quantity of buffer that is mixed into the quantity of pharmaceutical carrier. The quantity of buffer is used to ensure the present invention does not become too acidic or too basic in case any kind of additional substance is mixed into the present invention. The quantity of buffer can be, but is not limited to, a phosphate buffer, a glycerin/sodium-hydroxide buffer, a carbonate buffer, or a combination thereof.

The present invention could also used to provide ancillary therapeutic benefits to the patient after the present invention is administered to the patient. Thus, the present invention may further comprise a quantity of additional therapeutic agents that is mixed into the quantity of pharmaceutical carrier. The additional therapeutic agents are used to have some kind of medicinal benefit on the user after the present invention has been administered to the patent. The quantity of additional therapeutic agents can be, but is not limited to, at least one corticosteroid, at least one cytotoxic drug, at least one antibiotic, at least one immunosuppressant drug, at least one nonsteroidal anti-inflammatory drug, at least one narcotic analgesic, at least one local anesthetic, at least one N-Methyl-D-aspartate receptor antagonist, at least one neuroleptic, at least one anticonvulsant, at least one antispasmodic, at least one antiemetic, at least one antidepressant, at least one muscle relaxant, at least one benzodiazepine, at least one barbiturate, at least one alpha-2 adrenoreceptor agonist, and a combination thereof.

In contrast to dexmedetomidine HCl or midazolam HCl when administered separately/individually to a pediatric patient by any route, the present invention effectively: (1) prevents/treats PED; (2) improves hemodynamic stability during and after procedures requiring anesthetic care for pediatric patients; (3) decreases the need for additional medications to be administered (i.e. sedatives, induction agents, narcotics, anticholinergics, antisialogogues, etc.); (4) is concentrated in order to avoid increasing ingested volumes in the stomach and gastrointestinal (GI) tract; (5) provides patient relaxation, sedation, anxiolysis, and analgesia; (6) decreases separation anxiety; (7) increases cooperation and mask acceptance during induction of general anesthesia; (8) provides neuro-protective effects; and (9) decreases risks (including respiratory depression, airway obstruction, desaturation, PONV, prolonged LOS, delayed discharge, inpatient admission, patient injury, increased pain, medical device damage/dislodgement, hemorrhage, etc.). Overall, the present invention leads to greater patient satisfaction, decreased costs, and greater patient safety.

No other single agent available is able to do all of this when administered at any point in the surgical experience. However, the present invention is administered PO in the pre-operative/pre-surgical area(s), providing upfront/prophylactic coverage against any physiologic insults, ordinary and extraordinary, incurred during the surgical/anesthetic experience that may cause PED or other harm to the pediatric patient.

Furthermore, current pre-operative agents for sedation and/or analgesia are in separate single- and/or multi-dose vials/containers. Pre-operative staff draws up prescribed volumes, concentrations, and doses in a syringe or syringes for administration. Syringes are only labeled in “mL” increments, and not drug-specifically labeled. Furthermore, drawn up doses are vulnerable to human error by staff. The system for administering the present invention is a prefilled, all-in-one, pre-labeled syringe. Many pediatric patients will be turned off to further medications or additional medications/syringes after the first administer dosage. Thus, the system for administering the present invention can also be a prefilled, one-time/single-use syringe. This results in single drug administrations to pediatric patients, decreased chance of human error in dosing, and decreased risk of contamination from other drugs or breaks in sterile/clean techniques present during multi-dose drug vial dosing/administration.

Although the invention has been explained in relation to its preferred embodiment, it is to be understood that many other possible modifications and variations can be made without departing from the spirit and scope of the invention as hereinafter claimed.

What is claimed is:

1. A pre-operative composition for sedation or analgesia comprises:
a quantity of dexmedetomidine;
a quantity of midazolam;
a quantity of pharmaceutical carrier; and
the quantity of dexmedetomidine and the quantity of midazolam being mixed into the quantity of pharmaceutical carrier.

2. The pre-operative composition for sedation or analgesia as claimed in claim 1, wherein the quantity of dexmedetomidine is formulated as a salt.

3. The pre-operative composition for sedation or analgesia as claimed in claim 2, wherein the salt is further composed of hydrochloride.

4. The pre-operative composition for sedation or analgesia as claimed in claim 1, wherein the quantity of midazolam is formulated as a salt.

5. The pre-operative composition for sedation or analgesia as claimed in claim 4, wherein the salt is further composed of hydrochloride.

6. The pre-operative composition for sedation or analgesia as claimed in claim 1, wherein the quantity of dexmedetomidine ranges from a concentration of 2 micrograms per milliliter (mcg/mL) within the pre-operative composition to a concentration of 50 mcg/mL within the pre-operative composition.

7. The pre-operative composition for sedation or analgesia as claimed in claim 1, wherein the quantity of midazolam ranges from a concentration of 100 mcg/mL within the pre-operative composition to a concentration of 5000 mcg/mL within the pre-operative composition.

8. The pre-operative composition for sedation or analgesia as claimed in claim 1, wherein:
the quantity of dexmedetomidine is approximately at a concentration of 16 mcg/mL within the pre-operative composition; and
the quantity of midazolam is approximately at a concentration of 400 mcg/mL within the pre-operative composition.

9. The pre-operative composition for sedation or analgesia as claimed in claim 1, wherein the quantity of pharmaceutical carrier is selected from a group consisting of: water, ethanol, glycerin, polyethylene, glycol, propylene glycol, and a combination thereof.

10. The pre-operative composition for sedation or analgesia as claimed in claim 1 comprises:
a quantity of sweetening agent; and
the quantity of sweetening agent being mixed into the quantity of pharmaceutical carrier;

11. The pre-operative composition for sedation or analgesia as claimed in claim 10, wherein the quantity of sweetening agent is cyclodextrin.

12. The pre-operative composition for sedation or analgesia as claimed in claim 10, wherein the quantity of sweetening agent is approximately at a concentration of 50000 mcg/mL within the pre-operative composition.

13. The pre-operative composition for sedation or analgesia as claimed in claim 1 comprises:
a quantity of other flavoring agents; and
the quantity of other flavoring agents being mixed into the quantity of pharmaceutical carrier.

14. The pre-operative composition for sedation or analgesia as claimed in claim 13, wherein the quantity of other flavoring agents is selected from a group consisting of: at least one carbohydrate, at least one sweeting agent, at least one flavor enhancer, at least one preservative, at least one antifungal agent, and a combination thereof.

15. The pre-operative composition for sedation or analgesia as claimed in claim 1, wherein a pH level of the pre-operative composition is greater than or equal to 4.

16. The pre-operative composition for sedation or analgesia as claimed in claim 1 comprises:
a quantity of buffer; and
the quantity of buffer being mixed into the quantity of pharmaceutical carrier.

17. The pre-operative composition for sedation or analgesia as claimed in claim 16, wherein the quantity of buffer is selected from a group consisting of: a phosphate buffer, a glycerin/sodium-hydroxide buffer, a carbonate buffer, and a combination thereof.

18. The pre-operative composition for sedation or analgesia as claimed in claim 1 comprises:
a quantity of additional therapeutic agents; and
the quantity of additional therapeutic agents being mixed into the quantity of pharmaceutical carrier.

19. The pre-operative composition for sedation or analgesia as claimed in claim 18, wherein the quantity of additional therapeutic agents is selected from a group consisting of: at least one corticosteroid, at least one cytotoxic drug, at least one antibiotic, at least one immunosuppressant drug, at least one nonsteroidal anti-inflammatory drug, at least one narcotic analgesic, at least one local anesthetic, at least one N-Methyl-D-aspartate receptor antagonist, at least one neuroleptic, at least one anticonvulsant, at least one antispasmodic, at least one antiemetic, at least one antidepressant, at least one muscle relaxant, at least one benzodiazepine, at least one barbiturate, at least one alpha-2 adrenoreceptor agonist, and a combination thereof.
